(19) United States
(12) Patent Application Publication
(10) Pub. No.: US 2025/0057567 A1
(43) Pub. Date: Feb. 20, 2025

(54) MAPPING AND TRANSSEPTAL PUNCTURE CATHETER

(71) Applicant: Boston Scientific Scimed, Inc., Maple Grove, MN (US)

(72) Inventors: Jackie Leung, Richmond Hill (CA); Prateek Mathur, Toronto (CA)

(21) Appl. No.: 18/798,203

(22) Filed: Aug. 8, 2024

Related U.S. Application Data
(60) Provisional application No. 63/519,769, filed on Aug. 15, 2023.

(51) Int. Cl. A61B 17/34; A61B 17/00; A61B 18/00; A61B 18/14

(52) U.S. Cl. CPC A61B 17/3417; A61B 18/1487; A61B 2017/00044; A61B 2017/3454; A61B 2018/00363; A61B 2018/00839; A61B 2018/1467

(57) ABSTRACT

A medical system for performing a procedure within a heart of a patient includes an elongate member having a proximal portion and a pre-formed distal portion. The pre-formed distal portion is configured to contact a portion of a fossa ovalis of the heart. The elongate member includes a lumen extending from the proximal portion to a location proximal of the pre-formed distal portion. The system includes a perforating device having a proximal portion and a distal portion including a puncturing tip. The perforating device is configured to translate within the lumen. The puncturing tip is located substantially in the center of the pre-formed distal portion when the pre-formed distal portion is in contact with the fossa ovalis.

### CROSS REFERENCE TO RELATED APPLICATIONS

This application claims priority to U.S. Provisional Application No. 63/519,769 entitled “MAPPING AND TRANSSEPTAL PUNCTURE CATHETER,” filed Aug. 15, 2023, which is incorporated herein by reference in its entirety.

### TECHNICAL FIELD

The present invention relates generally to methods and devices usable within the body of a patient. More specifically, the present invention is concerned with a device having electroanatomical mapping (EAM) and transseptal crossing capabilities.

### BACKGROUND

Electroanatomical mapping (EAM) is an increasingly prevalent technology useful during in vivo procedures. It enables physicians to identify anatomical regions of the heart and patterns of electrical activation. This is especially useful when treating arrhythmias. Devices that are compatible with EAM systems allow operators to localize them and more easily target specific regions for treatment, allowing for better workflow, better treatment efficacy, and shorter procedure time.

In the treatment of atrial fibrillation and other electrophysiology (EP) procedures, physicians typically need to gain access to the left side of the heart by puncturing and crossing the fossa ovalis (FO) under fluoroscopic image guidance. With the introduction of electroanatomical mapping (EAM) techniques, fluoroscopy use can be reduced or eliminated. However, this comes at a cost of more complex devices and workflows. Primarily, an EAM guided transseptal puncture (TSP) requires that a map of the right atrium be generated before puncture device can be reliably positioned. The need for a mapping catheter as well as additional exchanges to access the left atrium is a substantial obstacle in the adoption of EAM for TSP.

### SUMMARY

Example 1 is a medical system for performing a procedure within a heart of a patient. The system includes an elongate member having a proximal portion and a pre-formed distal portion. The pre-formed distal portion is configured to contact a portion of a fossa ovalis of the heart. The elongate member includes a lumen extending from the proximal portion to a location proximal of the pre-formed distal portion. The system includes a perforating device having a proximal portion and a distal portion including a puncturing tip, the perforating device being configured to translate within the lumen. The puncturing tip is located substantially in the center of the pre-formed distal portion when the pre-formed distal portion is in contact with the fossa ovalis.

Example 2 is the system of Example 1, wherein pre-formed distal portion includes a loop, arch, or spiral.

Example 3 is the system of Example 1, wherein the elongate member includes a magnetic sensor configured to electrically couple with an EAM system.

Example 4 is the system of any of Examples 1-3, wherein the elongate member includes a plurality of electrodes configured to electrically couple with an EAM system for mapping a tissue surface or locating a tissue surface.

Example 5 is the system of Example 4, wherein the plurality of electrodes are spaced uniformly along the pre-formed distal portion.

Example 6 is the system of Example 4, wherein the plurality of electrodes are positioned in pairs along the pre-formed distal portion.

Example 7 is the system of any of Examples 1-6, wherein the elongate member proximal portion is configured to connect with an EAM system.

Example 8 is the system of any of Examples 1-7, wherein the pre-formed distal portion remains substantially linear in a constrained state, and forms a loop, arch, or spiral in an unconstrained state.

Example 9 is the system of any of Examples 1-8, wherein the puncturing tip is an RF electrode or a needle tip.

Example 10 is the system of any of Examples 1-9, further comprising a dilator including a dilator proximal portion, a tapered distal portion, and a dilator lumen extending between the dilator proximal portion and the tapered distal portion.

Example 11 is the system of Example 10, wherein the dilator lumen is configured to constrain the pre-formed distal portion of the elongate member.

Example 12 is the system of any of Examples 1-11, further comprising an outer hollow member configured to constrain the pre-formed distal portion of the elongate member.

Example 13 is the system of any of Examples 1-12, wherein the perforating device distal portion includes a curve.

Example 14 is the system of any of Examples 1-13, wherein the pre-formed distal portion is configured to identify the fossa ovalis.

Example 15 is the system of any of Examples 1-14, wherein the pre-formed distal portion forms a loop, arch, or spiral in an unconstrained state, and wherein the loop, arch, or spiral is in a single plane.

Example 16 is a medical system for performing a procedure within a heart of a patient including an elongate member having a proximal portion and a pre-formed distal portion. The pre-formed distal portion is configured to contact a portion of a fossa ovalis of the heart. A plurality of electrodes are located along the pre-formed distal portion, and a lumen extends from the proximal portion to a location proximal of the pre-formed distal portion. The system includes a perforating device having a proximal portion and a distal portion including a puncturing tip. The perforating device is configured to translate within the lumen. The puncturing tip is located substantially in the center of the pre-formed distal portion when the pre-formed distal portion is in contact with the fossa ovalis.

Example 17 is the system of Example 16, wherein pre-formed distal portion includes a loop, arch, or spiral.

Example 18 is the system of Example 16, wherein the elongate member includes magnetic sensor configured to electrically couple with an EAM system.

Example 19 is the system of Example 16, wherein the plurality of electrodes are configured to electrically couple with an EAM system.

Example 20 is the system of Example 19, wherein the plurality of electrodes are spaced uniformly along the pre-formed distal portion for mapping a tissue surface or locating a tissue surface.

Example 21 is the system of Example 19, wherein the plurality of electrodes are positioned in pairs along the pre-formed distal portion.

Example 22 is the system of Example 16, wherein the elongate member proximal portion is configured to connect with an EAM system.

Example 23 is the system of Example 16, wherein the pre-formed distal portion remains substantially linear in a constrained state, and forms a loop, arch, or spiral in an unconstrained state.

Example 24 is the system of Example 16, wherein the puncturing tip is an RF electrode or a needle tip.

Example 25 is the system of Example 16, further comprising a dilator including a dilator proximal portion, a tapered distal portion, and a dilator lumen extending between the dilator proximal portion and the tapered distal portion.

Example 26 is the system of Example 25, wherein the dilator lumen is configured to constrain the pre-formed distal portion of the elongate member.

Example 27 is the system of Example 16, further comprising an outer hollow member configured to constrain the pre-formed distal portion of the elongate member.

Example 28 is the system of Example 16, wherein the perforating device distal portion includes a curve.

Example 29 is the system of Example 16, wherein the pre-formed distal portion is configured to identify the fossa ovalis.

Example 30 is the system of Example 16, wherein the pre-formed distal portion forms a loop, arch, or spiral in an unconstrained state, and wherein the loop, arch, or spiral is in a single plane.

Example 31 is a method for providing access to the left atrium of a heart. The method includes advancing an elongate member having a pre-formed distal portion into a right atrium of the heart. A portion of a fossa ovalis of the heart is contacted with the pre-formed distal portion. A perforating device is advanced through a lumen of the elongate member, wherein a puncturing tip of the perforating device is located substantially in the center of the pre-formed distal portion when the pre-formed distal portion is in contact with the fossa ovalis. The method includes piercing the fossa ovalis with the puncturing tip.

Example 32 is the method of Example 31, further comprising mapping the right atrium using a plurality of electrodes on the pre-formed distal portion of the elongate member, wherein the plurality of electrodes are electrically coupled to an EAM system.

Example 33 is the method of Example 31, further comprising dilating the pierced fossa ovalis.

Example 34 is a medical system for performing a procedure within a heart of a patient including an elongate member having a proximal portion and a pre-formed distal portion. The pre-formed distal portion is configured to form a loop, arch, or spiral and to contact a portion of a fossa ovalis of the heart. A lumen extends from the proximal portion to a location proximal of the pre-formed distal portion. The system includes a perforating device having a proximal portion and a distal portion including a puncturing tip. The perforating device is configured to translate within the lumen. The system includes a dilator having a dilator proximal portion, a tapered distal portion, and a lumen extending between the dilator proximal portion and the tapered distal portion. The puncturing tip is located substantially in the center of the pre-formed distal portion when the pre-formed distal portion is in contact with the fossa ovalis.

Example 35 is the system of Example 34, wherein the dilator lumen is configured to constrain the pre-formed distal portion of the elongate member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIGS. 2A and 2B illustrate a device having EAM and transseptal crossing capabilities in accordance with an embodiment of the present disclosure.

FIG. 3 Illustrates the device of FIGS. 2A and 2B deployed in a right atrium of a patient's heart.

FIG. 4 illustrates the device of FIGS. 2A and 2B during a portion of a transeptal crossing procedure in accordance with an embodiment of the present disclosure.

FIG. 5 illustrates a portion of a transeptal crossing procedure in accordance with an embodiment of the present disclosure.

FIG. 6 illustrates a portion of a transeptal crossing procedure in accordance with an embodiment of the present disclosure.

FIG. 7 illustrates a device having EAM and transseptal crossing capabilities during a portion of a transeptal crossing procedure in accordance with an embodiment of the present disclosure.

FIG. 8 illustrates a portion of a transeptal crossing procedure in accordance with an embodiment of the present disclosure.

FIG. 9 illustrates a portion of a transeptal crossing procedure in accordance with an embodiment of the present disclosure.

FIG. 10 illustrates a portion of a transeptal crossing procedure in accordance with an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

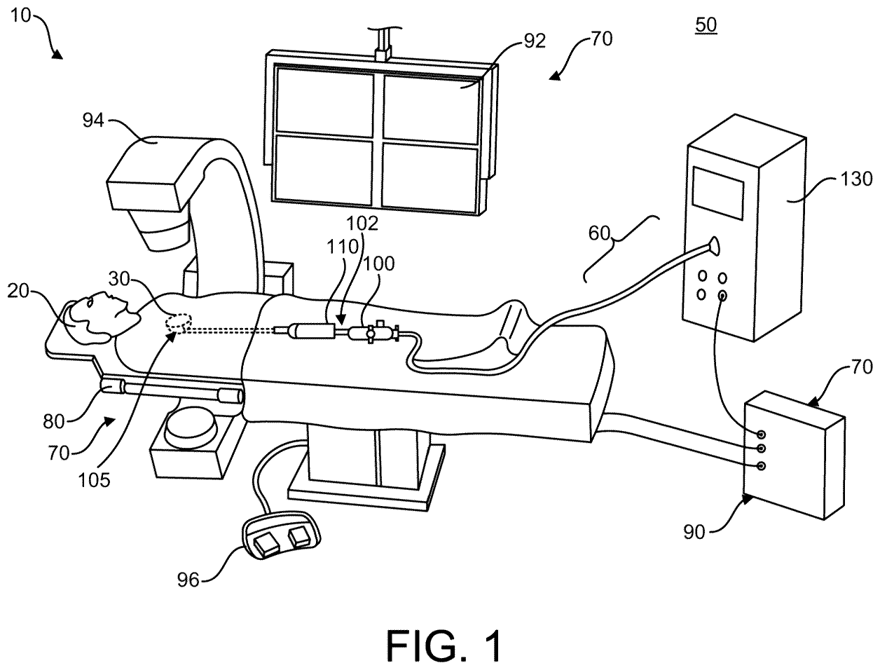

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an access system 60 and an EAM system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The access system 60 includes a dilator 100 having a proximal portion 102 and a distal portion 105, an introducer sheath 110, and a console 130. In some aspects, the distal portion 105 includes a tapered region. Additionally, the access system 60 includes various connecting elements, e.g., cables, umbilicals, and the like, that operate to functionally connect the components of the access system 60 to one another and to the components of the EAM system 70. This arrangement of connecting elements is not of critical importance to the present disclosure, and the skilled artisan will recognize that the various components described herein can be interconnected in a variety of ways.

In some embodiments, the introducer sheath 110 is operable to provide a delivery conduit through which the dilator 100, in particular all or part of the distal portion 105 thereof, can be deployed to the specific target sites within the patient's heart 30. In some aspects, the sheath 110 provides a delivery conduit for medical devices other than the dilator 100. The dilator 100 is configured with a lumen such that a guiding device, for example a guide wire, or perforation device, for example an RF perforation device, can be inserted therein. In some aspects, a perforation device can be used to perform a transseptal crossing procedure within the heart 30. It is understood that different workflows may necessitate different order of operations for positioning of components, for example the introducer sheath, 110, dilator 100, and perforation device inside the body at any given time. For example, in some procedures the dilator 100 may or may not be present during access to the patent's heart 30.

The console 130 is configured to control functional aspects of the access system 60. In embodiments, the console 130 includes one or more controllers, microprocessors, and/or computers that execute code out of memory to control and/or perform the functional aspects of the access system 60. In embodiments, the memory can be part of the one or more controllers, microprocessors, and/or computers, and/or part of memory capacity accessible through a network, such as the world wide web. In embodiments, the console 130 can include pulse generator hardware, software and/or firmware configure to generate electrical pulses in predefined waveforms, which can be transmitted to electrodes positioned on the dilator 100, guiding device, or perforation device to generate electric fields sufficient to achieve the desired clinical effect, for example ablation of target tissue through irreversible electroporation. In embodiments, the console 130 can deliver the pulsed waveforms in a monopolar or bipolar mode of operation, as will be described in further detail herein.

The EAM system 70 is operable to track the location of the various functional components of the access system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the dilator 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the dilator 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

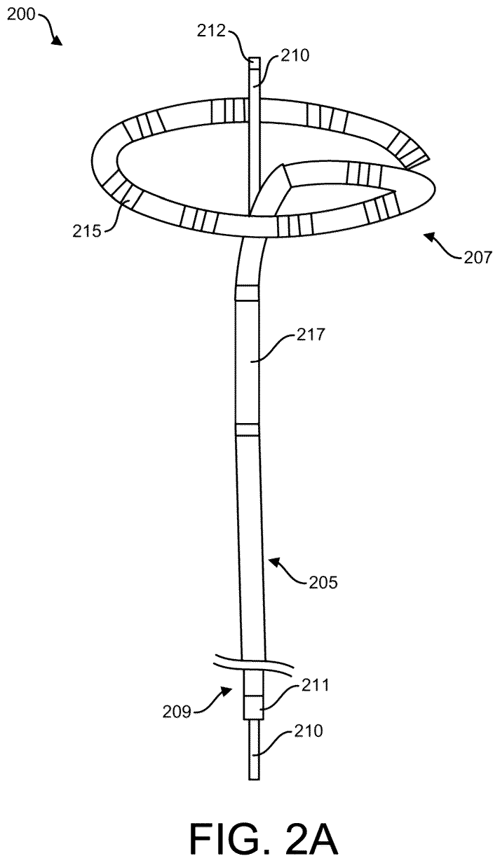

FIGS. 2A and 2B illustrate a system 200 having EAM and transseptal crossing capabilities in accordance with an embodiment of the present disclosure. The system 200 includes a catheter 205 including an elongate body having a pre-formed distal portion 207 and a proximal portion 209. The proximal portion 209 includes a connector 211 capable of electrically connecting the catheter 205 to the EAM system 70.

The pre-formed distal portion 207 is configured to create a loop or circle in an unconstrained state. In some embodiments, the pre-formed distal portion 207 is configured to form a spiral. In some embodiments, the pre-formed distal portion 207 is configured to form an arch. The pre-formed distal portion 207 remains in a substantially linear configuration when constrained, for example by a lumen. In some embodiments, the shape of the pre-formed distal portion 207 can be changed or modified by one or more steering wire or push rod. For example, the size of the loop, circle, arch, or spiral may be adjustable by actuating the one or more steering wire or push rod.

The pre-formed distal portion 207 includes a distal tip 213. Arranged along the pre-formed distal portion 207 is a plurality of electrodes 215. In some embodiments, the plurality of electrodes 215 are arranged in uniformly spaced pairs along the pre-formed distal portion 207. In some embodiments, the plurality of electrodes 215 are evenly spaced along the pre-formed distal portion 207. In some embodiments, the plurality of electrodes 215 extend proximally from the preformed distal portion 207 along the elongate body of the catheter 205. The plurality of electrodes 215 are configured to electrically connect to the EAM system 70 via the connector 211 for mapping portions of a patient's heart. In some aspects, the connector 211 may include a cable or be configured to connect to a cable. The catheter 205 also includes a magnetic sensor 217 located proximal of the pre-formed distal portion 207.

The pre-formed distal portion 207 is configured for pressing against the compliant wall of the FO in order to distinguish it from surrounding firm tissue. This allows for a high level of confidence in knowing the location of the FO. The loop of the pre-formed distal portion enables tenting of the FO that is easily identifiable on the EAM system 70. The EAM system 70 is capable of mapping the various surfaces of the heart.

The catheter 205 includes a lumen that extends from the proximal portion 209 and extends to an opening 219 that is positioned proximal the pre-formed distal portion 207. The lumen is configured to allow for fluids or medical devices to be released from or advanced out of the opening 219. The opening 219 is positioned such that it lies in the center of the pre-formed distal portion 207. As such, while the pre-formed distal portion 207 is pressing against the FO and tenting the tissue, a medical device that extends from the opening 219 will be located at the geometric center of the structure created by the pre-formed distal end 207. In some embodiments, the opening 219 is positioned such that it lies off center of the pre-formed distal portion 207. In this configuration, while the pre-formed distal portion 207 is pressing against the FO and tenting the tissue, a medical device that extends from the opening 219 will be located away from the geometric center of the structure created by the pre-formed distal end 207.

In one embodiment, a perforation device 210 is configured to be advanced through the lumen and out of the opening 219. The perforation device 210 includes a distal RF electrode 212 that is configured to be energized in order to puncture tissue, for example a patent's FO during a transseptal puncture procedure. The perforation device 210 includes a proximal portion that is capable of electrically connecting with an RF system to selectively energize the distal RF electrode 212. In some embodiments, the lumen includes an extendable nose to help support the perforation device 210 (e.g., an RF puncture wire) during puncture of the FO. This could include, for example, a hypotube that is capable of extending out of the lumen to support the perforation device 210. In some embodiments, the extendable hypotube is configured with a sharp tip capable of puncturing tissue. In some embodiments, the perforation device 210 may include a needle having a sharp tip as opposed to a distal RF electrode 212.

In some embodiments, the pre-formed distal portion 207 is formed as a balloon. The balloon can include a shape configured to press against the FO and place one or more electrodes against the FO. The balloon is configured with a central lumen though which the perforation device 210 extends to pierce the target tissue.

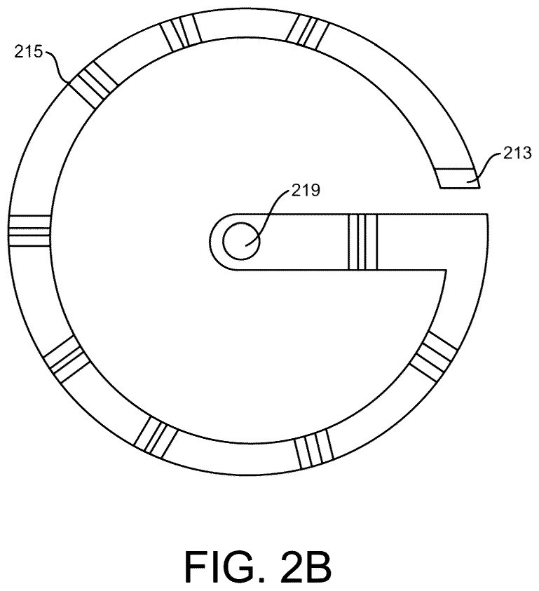

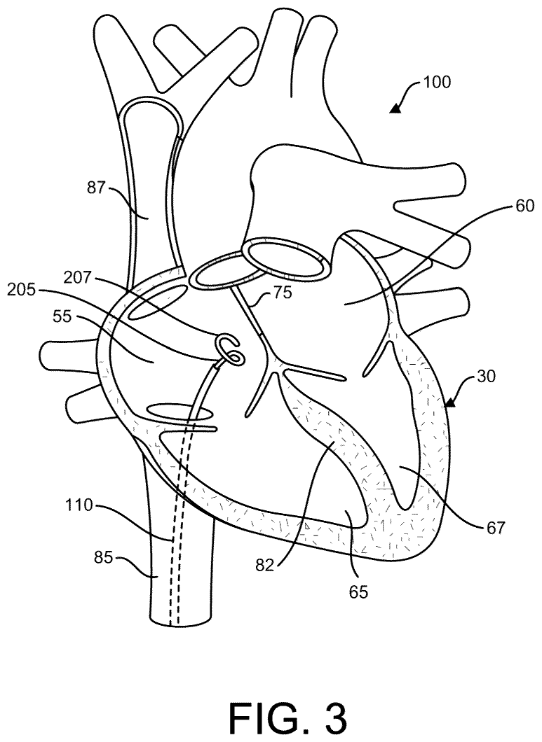

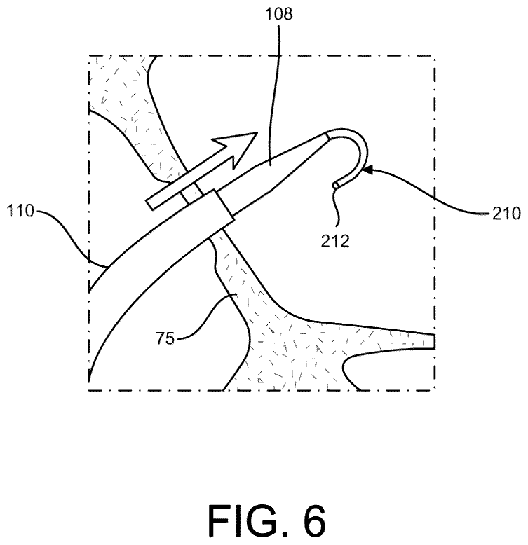

FIGS. 3-6 are schematic illustrations of a medical procedure 100 within a patient's heart 30 utilizing a system 200 according to embodiments of the disclosure. As is known, the human heart 30 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 67 is a ventricular septum 82. As is further known, deoxygenated blood from the patient's body is returned to the right atrium 55 via an inferior vena cava (IVC) 85 or a superior vena cava (SVC) 87.

The medical procedure 10 illustrated in FIGS. 3-6 is an exemplary embodiment for providing access to the left atrium 60 using the system 200 for subsequent deployment of diagnostic and/or therapeutic devices within the left atrium 60. As shown in FIGS. 3-6, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the system 200 is advanced into the right atrium 55 via the SVC 87.

In the illustrated embodiment, the system 200 includes an introducer sheath 110, a dilator 100 having a tapered distal tip portion 108, a radiofrequency (RF) perforation device 210, also known as a piercing device, having distal end portion terminating in a tip electrode 212, and a catheter 205 having a pre-formed distal portion 207. As shown, in the assembled use state illustrated in FIGS. 3-6, the RF perforation device 210 can be disposed within the catheter 205. In one embodiment in which the system 200 is deployed into the right atrium 55 via the IVC 85, a user introduces a guidewire (not shown) into a femoral vein, typically the right femoral vein, and advances it towards the heart 30. The sheath 110 may then be introduced into the femoral vein over the guidewire, and advanced towards the heart 30. In one embodiment, the distal ends of the guidewire and sheath 110 are then positioned in the SVC 87. These steps may be performed with the aid of an imaging system, e.g., fluoroscopy or ultrasonic imaging. The catheter 205 may then be introduced into the sheath 110 and through the sheath 110 into the SVC 87. Alternatively, the catheter 205 may be fully inserted into the sheath 110 prior to entering the body, and both may be advanced simultaneously towards the heart 30.

Subsequently, the user may extend the catheter 205 from the sheath 110 such that the pre-formed distal portion 207 achieves the unconstrained mapping configuration, for example a loop, arch, or spiral. A map of the right atrium can be made using the plurality of electrodes 215. The user may then position the pre-formed distal portion 207 against the atrial septum 75 in order to locate the fossa ovalis for puncturing or for mapping the tissue surface. The user may tent the tissue using the pre-formed distal portion 207. The RF perforation device 210 is then extended through the lumen of the catheter 205 and out of the opening 219 such that electrode 212 is located substantially at the center of the pre-formed distal portion 207 and is aligned with the target site for puncturing.

With the tip electrode 212 positioned at the target site, energy is delivered from an energy source, e.g., an RF generator, through the RF perforation apparatus 210 to the tip electrode 212 and the target site. In some embodiments, the energy is delivered at a power of at least about 5 W at a voltage of at least about 75 V (peak-to-peak), and functions to vaporize cells in the vicinity of the tip electrode 212, thereby creating a void or perforation through the tissue at the target site. The user then applies force to the RF perforation device 210 so as to advance the tip electrode 212 at least partially through the perforation. In these embodiments, when the tip electrode 212 has passed through the target tissue, that is, when it has reached the left atrium 60, energy delivery is stopped. In some embodiments, the step of delivering energy occurs over a period of between about 1 s and about 5 s.

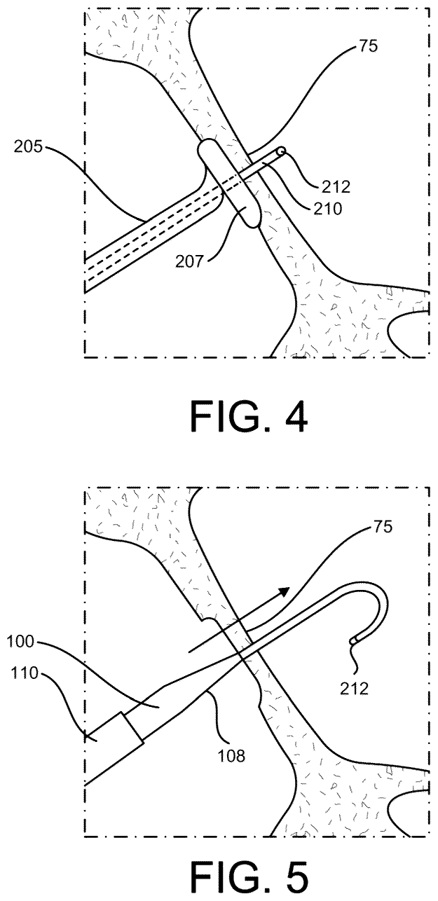

With the tip electrode 212 of the RF perforation device 210 having crossed the atrial septum 75, the catheter 205 can be withdrawn from the sheath 110. In some embodiments, the catheter 205 may include a feature, such as a score line or perforation, that allows the catheter to be torn from the RF perforation device 210. After removing the catheter 205, the dilator 100 can be advanced through the sheath 110 into the right atrium 55. Subsequently, the dilator 100 can be advanced forward, as indicated by the arrow in FIG. 5, with the tapered distal tip portion 108 operating to gradually enlarge the perforation made by the tip electrode 212 to permit advancement of the distal end of the sheath 110 into the left atrium 60.

In some embodiments, the distal end portion of the RF perforation device 210 may be pre-formed to assume an atraumatic shape such as a J-shape (as shown in FIGS. 3-6), a pigtail shape or other shape selected to direct the tip electrode 212 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 212 and tissue within the left atrium 60 and can also operate to anchor the distal end portion within the left atrium 60 during subsequent procedural steps. For example, in embodiments, the RF perforation device 210 can be structurally configured to function as a delivery rail for deployment of a relatively larger bore therapy delivery sheath and associated dilator(s). In such embodiments, the dilator 100 and the sheath 110 are withdrawn following deployment of the distal end portion of the RF perforation device 210 into the left atrium 60. The anchoring function of the pre-formed distal end portion inhibits unintended retraction of the distal end portion, and corresponding loss of access to the perforated site on the atrial septum 75, during such withdrawal.

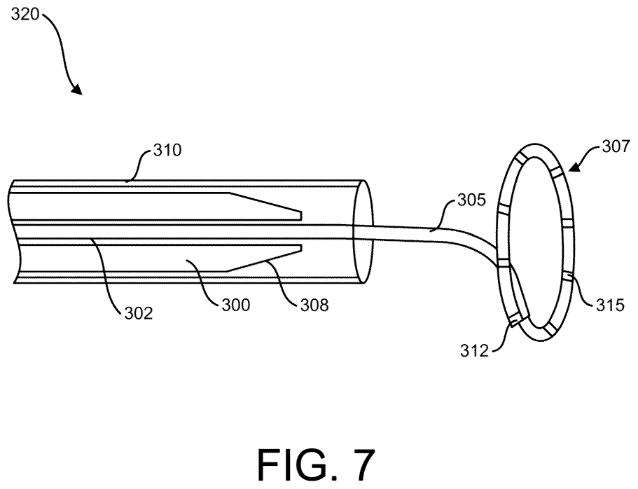

FIGS. 7-10 illustrate a system 320 having EAM and transseptal crossing capabilities in accordance with an embodiment of the present disclosure. The system 320 includes an introducer sheath 310, a dilator 300 having a tapered portion 308, and a catheter 305 having a pre-formed distal portion 307. The catheter 305 includes a plurality of electrodes 315 configured to electrically connect to an EAM system 70 as discussed above as well as a distal RF electrode 312. The dilator 300 includes a lumen 302 through which the catheter 305 is configured to translate. The lumen 302 is configured to constrain the pre-formed distal portion 307 of the catheter 305 while the system 320 is advanced through a patient's vasculature.

FIG. 7 illustrates the system 320 in a mapping configuration. In FIG. 7 the system 320 can be located at a desired location within a patient's heart, such as the right atrium. In one embodiment, the system 320 may be advanced together to the desired location. In another embodiment, the sheath 310 can be advanced to the desired location along a guidewire or other guiding member. Following removal of the guidewire or guiding member, the dilator 300, with or without the catheter 305, can be advanced though the sheath 310 to the location. If not advanced with the dilator 300, the catheter 305 can then be advanced through the lumen 302 of the dilator 300 to the location.

Once at the desired location, for example the right atrium, the catheter 305 is extended from the dilator 300 and the sheath 310 such that the pre-formed distal portion 307 assumes the pre-formed curve. In the mapping configuration, the pre-formed distal portion 307 can be placed against a target tissue in order to locate a portion of the target tissue or map various tissue surfaces. In one embodiment, the target tissue is the fossa ovalis (FO) as discussed above.

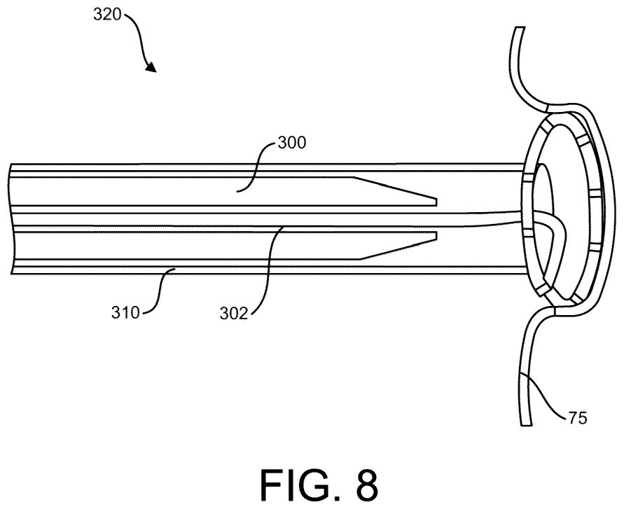

FIG. 8 illustrates the introducer sheath 310 being advanced after the FO has been identified by the catheter 305. The introducer sheath 310 can aid in providing force to tent the tissue of the atrial septum 75 as well as ensuring that the identified tissue is not lost.

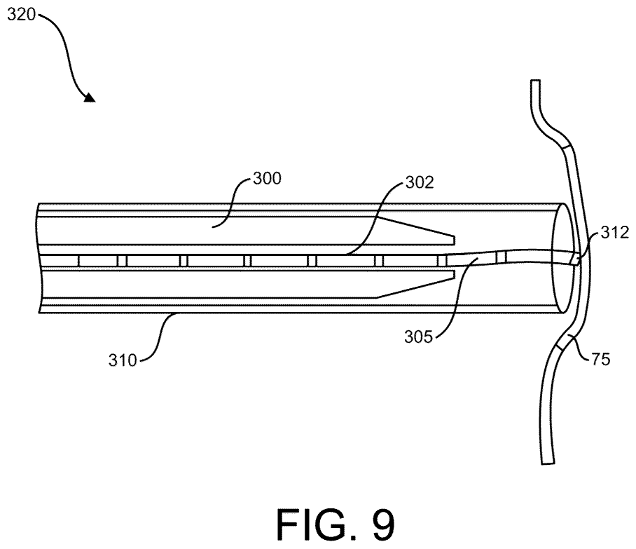

Once the distal end of the introducer sheath 310 is pressed against the target tissue, the catheter 305 can be withdrawn into the dilator 300. FIG. 9 illustrates the catheter 305 being withdrawn into the introducer sheath 310 and the dilator 300. The pre-formed distal portion 307 is constrained by the lumen 302 such that the pre-formed distal portion 307 is substantially linear. The catheter 305 is withdrawn until the distal RF electrode 312 can be positioned against the target tissue.

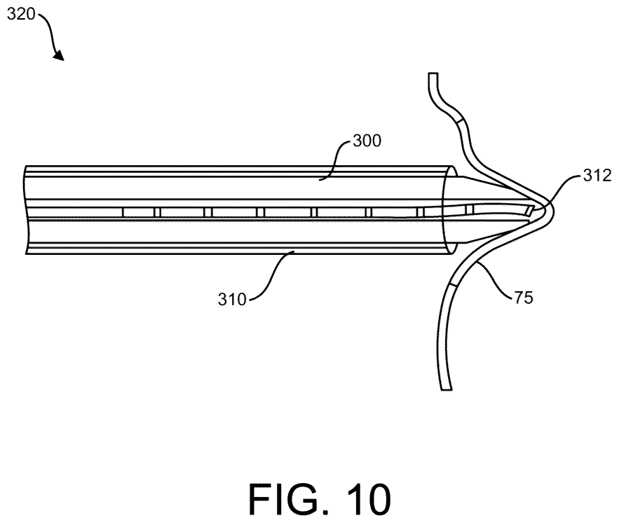

While holding the sheath 310 stationary, the dilator 300 is advanced to the target tissue. As shown in FIG. 10, the dilator 300 is advanced simultaneously or just after energy is delivered to the distal RF electrode 312. This allows for puncturing the FO with the distal RF electrode 312 and stretching of the puncture with the tapered portion 308 of the dilator 300 such that the sheath 310 can be advanced into the left atrium.

In some embodiments, the sheath 310, dilator, 300, and/or catheter 305 include a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the sheath 310, dilator, 300, and/or catheter 305. In some embodiments, there is a single cut that winds around an axis with a wider spacing between loops at the proximal portion and a larger spacing at the distal portion. The spacing and size of the cuts can be varied to achieve different flexibilities along the length of the sheath 310, dilator, 300, and/or catheter 305.

In some embodiments, the pre-formed distal portion 307 of the catheter 305 is adjustable. The shape of the pre-formed distal portion 307 is changed using one or more pull or push wires, or by forming the pre-formed distal portion 307 of a shape memory material such as a shape memory polymer or a shape memory metal. This would allow the pre-formed distal portion to have a first shape at a first temperature, and a second shape at a second temperature. Shape transition may be initiated by inserting a heated solution into the catheter 305 or using electricity to heat a portion of the catheter 305.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system for performing a procedure within a heart of a patient, the system comprising:
an elongate member having a proximal portion and a pre-formed distal portion, the pre-formed distal portion being configured to contact a portion of a fossa ovalis of the heart, a plurality of electrodes located along the pre-formed distal portion, and a lumen extending from the proximal portion to a location proximal of the pre-formed distal portion; and
a perforating device having a proximal portion and a distal portion including a puncturing tip, the perforating device being configured to translate within the lumen;
wherein the puncturing tip is located substantially in the center of the pre-formed distal portion when the pre-formed distal portion is in contact with the fossa ovalis.

2. The system of claim 1, wherein pre-formed distal portion includes a loop, arch, or spiral.

3. The system of claim 1, wherein the elongate member includes a magnetic sensor configured to electrically couple with an EAM system.

4. The system of claim 1, wherein the plurality of electrodes are configured to electrically couple with an EAM system for mapping a tissue surface or locating a tissue surface.

5. The system of claim 4, wherein the plurality of electrodes are spaced uniformly along the pre-formed distal portion.

6. The system of claim 4, wherein the plurality of electrodes are positioned in pairs along the pre-formed distal portion.

7. The system of claim 1, wherein the elongate member proximal portion is configured to connect with an EAM system.

8. The system of claim 1, wherein the pre-formed distal portion remains substantially linear in a constrained state, and forms a loop, arch, or spiral in an unconstrained state.

9. The system of claim 1, wherein the puncturing tip is an RF electrode or a needle tip.

10. The system of claim 1, further comprising a dilator including a dilator proximal portion, a tapered distal portion, and a dilator lumen extending between the dilator proximal portion and the tapered distal portion.

11. The system of claim 10, wherein the dilator lumen is configured to constrain the pre-formed distal portion of the elongate member.

12. The system of claim 1, further comprising an outer hollow member configured to constrain the pre-formed distal portion of the elongate member.

13. The system of claim 1, wherein the perforating device distal portion includes a curve.

14. The system of claim 1, wherein the pre-formed distal portion is configured to identify the fossa ovalis.

15. The system of claim 1, wherein the pre-formed distal portion forms a loop, arch, or spiral in an unconstrained state, and wherein the loop, arch, or spiral is in a single plane.

16. A method for providing access to the left atrium of a heart, the method comprising:
advancing an elongate member having a pre-formed distal portion into a right atrium of the heart;
contacting a portion of a fossa ovalis of the heart with the pre-formed distal portion;
advancing a perforating device through a lumen of the elongate member, wherein a puncturing tip of the perforating device is located substantially in the center of the pre-formed distal portion when the pre-formed distal portion is in contact with the fossa ovalis; and
piercing the fossa ovalis with the puncturing tip.

17. The method of claim 16, further comprising mapping the right atrium using a plurality of electrodes on the pre-formed distal portion of the elongate member, wherein the plurality of electrodes are electrically coupled to an EAM system.

18. The method of claim 16, further comprising dilating the pierced fossa ovalis.

19. A medical system for performing a procedure within a heart of a patient, the system comprising:
an elongate member having a proximal portion, a pre-formed distal portion, and a puncturing tip, the pre-formed distal portion being configured to form a loop, arch, or spiral and to contact a portion of a fossa ovalis of the heart; and
a dilator including a dilator proximal portion, a tapered distal portion, and a lumen extending between the dilator proximal portion and the tapered distal portion;
wherein the dilator lumen is configured to constrain the pre-formed distal portion of the elongate member.

20. The system of claim 19, wherein the puncturing tip includes an RF electrode.
